(19) United States
(12) Patent Application Publication
(10) Pub. No.: US 2014/0110360 A1
(43) Pub. Date: Apr. 24, 2014

(54) DEVICE FOR THERMALLY TREATING PRODUCTS WITH CLEANING OF THE PROCESS LIQUID

(71) Applicant: KRONES AG, Neutraubling (DE)

(72) Inventors: Paul Braun, Kropp (DE); Jan Karsten Munzer, Friedrichstadt (DE)

(73) Assignee: KRONES AG, Neutraubling (DE)

(21) Appl. No.: 14/059,492

(22) Filed: Oct. 22, 2013

(30) Foreign Application Priority Data
Oct. 22, 2012 (DE) 10 2012 219 184.4

(51) Int. Cl. B01D 35/02; A61L 2/04

(52) U.S. Cl. CPC B01D 35/02; A61L 2/04
USPC 210/805; 210/167.01; 210/167.32

(57) ABSTRACT

A device for thermally treating products in containers, including supply and discharge conveyors for containers; with at least one irrigation or spraying zone for irrigating or spraying the containers with a process liquid, for example water; and a circulation circuit for at least partially reusing said process liquid, the circulation circuit including at least one pump; with at least one separation unit with several essentially parallel lamellae for cleaning the process liquid from particles, for example broken glass and/or sand, and the pump pumps the process liquid along the lamella.

### CROSS-REFERENCE TO RELATED APPLICATION

The present application claims the benefit of priority of German Application No. 10 2012 219 184.4, filed Oct. 22, 2012. The entire text of the priority application is incorporated herein by reference in its entirety.

### FIELD OF THE DISCLOSURE

The present disclosure relates to a device for thermally treating products with cleaning of the process liquid, such as used in bottling operations.

### BACKGROUND OF THE DISCLOSURE

Devices for the thermal treatment of products in containers, for example bottles, PET containers and cans, are well-known in prior art. These devices may comprise, for example, pasteurization devices, heating or cooling devices. These devices often also occur in combination in a large, multi-zone pasteurization device to bring the products at least temporarily to different defined temperature levels. One example is the SHIELD pasteurization tunnel by Krones. Heat exchange is generally effected by irrigation with a process liquid, for example water. Here, the term irrigation means the irrigation or spraying of the containers. The used water is normally at least partially reused. A recirculation operation of the process water takes place. Besides chemical water treatment, a mechanical filtration of the water is performed before it may be used again.

For separating particles, for example broken glass, sand and/or suspended matter, from the process water, travelling screens are typically employed. A cross-zone travelling screen, as it is employed in many cases, has the disadvantage that the belt must be guided horizontally for this, and correspondingly, the essentially vertical water path results in an additional machine height. This additional height may amount to, for example, up to 400 mm and involve considerable extra efforts and costs in the region of the conveyor means for supplying and discharging the containers. In case of a separation in zones by one travelling screen each per zone, the costs incurred for separation are disproportionally high. In simpler machines, plug-in screens are also used.

Both types of screen have the additional disadvantage that they may get clogged, for example due to suspended matter, slimy matter and substances swimming on the water. This means that these screens may get choked. The mentioned substances deposit, for example, on the screen surfaces and accumulate there. There, these substances increasingly hinder the throughput through the screens. The screens must be regularly checked for clogging and cleaned. In particular if plug-in screens are used, the plug-in screens must be possibly pulled out and cleaned, so that machines with plug-in screens involve a high amount of required work and/or personnel. Typically, suspended matter, slimy matter and substances swimming on the water are dissolved by chemical treatment. However, if one does not succeed, for example, in dissolving slimy matter or similar substances by chemical treatment, or if there are some troubles with the chemical water treatment, the screen surfaces of the mentioned screen types will get clogged all the faster.

The mesh size of a screen restricts the achievable flow rate. Typically, particles of a size of at most 2-3 mm may be filtered with a travelling screen or a plug-in screen. Acicular broken glass may possibly still pass through such meshes which may then lead to a clogging of the nozzles for irrigation. Furthermore, both the nozzles and impellers are often made of plastics. However, plastic nozzles may wear down from inside by broken glass or other solid substances of a diameter of less than 2-3 mm. However, a regular exchange of the nozzles is not desired, and neither is uncontrolled splashing, for example by water escaping laterally from ragged or damaged nozzles.

### SUMMARY OF THE DISCLOSURE

In view of the above mentioned problems, it is one aspect of the present disclosure to provide a separation option with a high separation efficiency and low risk of clogging for an above cited device.

### DESCRIPTION OF THE DISCLOSURE

The above mentioned aspect is achieved by a device for thermally treating products. In accordance with the disclosure, this is a device for thermally treating products in containers, and includes supply and discharge conveyors for containers; at least one treatment zone for irrigation or spraying the containers with a process liquid, for example water; and a circulation circuit for at least partially reusing the process liquid, the circulation circuit including at least one pump; and wherein at least one separation unit is provided with several essentially parallel lamellae for cleaning the process liquid from particles, for example broken glass and/or sand, the pump pumping the process liquid along the lamella.

The separation unit utilizes sedimentation by gravity in particular for small particles and suspended matter which are carried along in the process liquid. Treatment takes place by irrigating or spraying the container from outside. If several treatment zones are used, these may have different temperatures. The term treatment zone may be a synonym for irrigation zone or spraying zone or temperature zone. The process liquid is in particular meant to be water, also referred to as process water. However, it is possible to use other process liquids or mixtures. For cleaning, that means in particular for separating the particles from the process liquid, the difference in density between the process liquid and the undesired particles to be separated is utilized. The lamellae provide a large sedimentation area in a compact form which is passed by by the process liquid. The flow of liquid may be supported by pumping by means of a pump. The particles may sediment at the lamellae, that means at the sedimentation surfaces, and then they sink downwards in the liquid by gravity. It will be understood that the delivery rate of the pump may be selected such that the sinking particles are not entrained by a flow.

In the device, the lamellae may be provided underneath the liquid surface and be completely wetted by the process liquid.

The lamellae may be completely wetted, that means they may be provided to be immerged in the liquid. Thereby, the soiling of the lamellae's surfaces may be clearly reduced, and the lamellae remain practically clean from any residues. Thus, no residues may dry on the lamellae.

In the device, the lamellae may be provided obliquely to the horizontal line at an angle a, where preferably 30°<α<60°.

The inclination of the lamellae permits, for example, to provide flow resistance with respect to the direction of flow of the process liquid. Here, the angle at which the lamellae are adjusted to the horizontal line may be selected.

In the device, the lamellae may be exchangeable individually or in the form of lamella packs comprising several lamellae.

The lamellae or lamella packs, i.e. groups of lamellae, may be serviced or exchanged with little effort. For example, the lamellae or lamella packs may be insertable from above or from the side.

In the device, the lamellae may comprise an essentially liquid-tight surface, the surface being preferably coated.

Due to their surface properties, the lamellae may act as sedimentation surfaces and thus exactly not as screens. Thereby, the porosity of the surfaces may be kept preferably low. The accumulation of organic suspended matter, such as slime, at the lamellae may be also clearly reduced or even avoided thereby. Even if only few of such substances would accumulate at the surfaces of the lamellae, they would practically disturb none of the sedimentation properties of the lamellae. By this, an advantage over screen-like inserts may be achieved.

In the device, the separation unit may furthermore comprise an exchangeable screen box at the bottom of the separation unit for collecting the particles.

A screen box of an adequate size may be provided. The screen box may typically be discharged periodically. This discharge may be accomplished manually or automatically.

In the device, the separation unit may furthermore comprise an overflow edge via which cleaned process liquid will get into the circulation circuit again.

By the overflow edge, an edge in addition to the lamellae may be provided over which the process liquid must rise before it will return to the circulation circuit. In the process, the liquid rises essentially vertically, whereby an additional barrier for particles which have not yet sedimented at the lamellae may result.

The device may comprise several treatment zones, where the at least one separation unit may be associated with several zones.

The device may comprise several treatment zones, where one separation unit as described above may be associated with each zone.

In the device, one separation unit may be provided for several zones, or each zone may have its own separation unit. Correspondingly, several pumps may be provided for pumping the process liquid of different zones separately or for connecting the circulation circuits of several zones by means of pumps.

The device may be a pasteurization device, a heating device, or a cooling device.

The disclosure furthermore includes the use of a separation unit for cleaning a process liquid from particles, for example broken glass and/or sand, in a device for the thermal treatment of products in containers, the device having supply and discharge conveyors for containers; at least one treatment zone for irrigating or spraying the containers with the process liquid, for example water; and with a circulation circuit for at least partially reusing the process liquid, the circulation circuit comprising at least one pump; wherein the separation unit comprises several, essentially parallel lamellae, the pump pumping the process liquid along the lamellae.

So, a robust and inexpensive provision of a separation unit is permitted which involves relatively little maintenance requirements. The separation efficiency of this separation unit may provide clearly better separation performances than in screen-like units, such as a travelling screen or a plug-in screen. Moreover, the separation performance is practically not affected by the particle size. The lower risk of clogging of the machine thus reduces wear on nozzles, pumps or other installations of a device for the thermal treatment of products. It is also conceivable that devices or machines that use process water to be cleaned may be retrofitted with such a separation unit.

### BRIEF DESCRIPTION OF THE DRAWINGS

Below, embodiments of the disclosure will be described with reference to the drawings. The described embodiments are in each respect to be considered only as illustrative and not as restrictive, and various combinations of the stated features are included in the disclosure.

FIG. 1 shows a part of a pasteurization device consisting of three treatment zones.

FIG. 2 shows a treatment zone of a pasteurization device with a separation unit according to the present disclosure.

FIG. 3 shows a side view of the separation unit of FIG. 2 according to the present disclosure.

FIG. 4 shows a schematic diagram with a perspective view of a separation unit 1 as it is also shown in FIG. 3 taking into consideration the flow of the process liquid.

### DETAILED DESCRIPTION OF THE PREFERRED EMBODIMENT

FIG. 1 shows, by way of example, a pasteurization device 200 with several treatment zones 203.1, 203.2, 203.3, as they are well-known in prior art. Containers, such as bottles with reference numeral 211, are transported through on a conveyor belt 204, for example from the left to the right in FIG. 1. Treatment zones 203.1, 203.2 and 203.3 may have different temperatures, i.e. the containers 211 are irrigated or sprayed from outside with process water of different temperatures. FIG. 1 shows irrigation devices 213.1, 213.2 and 213.3. In this example, the containers 211 are irrigated or sprayed from above. However, it is also possible to spray the containers 211 from the side. The process water used in each treatment zone runs downwards from the containers 211 and collects in the collecting tanks 215.1, 215.2 and 215.3. The process water may be pumped again to the irrigation devices 213.1, 213.2, 213.3 by pumps (not shown) via pipes 217.1, 217.2 and 217.3 and may be reused. Before the process water is used again, it may be cleaned mechanically by means of travelling screens or plug-in screens (not shown) in the respective collecting tanks 215.1, 215.2 and 215.3, or be pumped through screening stations (not shown). A chemical cleaning of the process water from organic substances is also possible. The treatment zones 203.1, 203.2 and 203.3 may be interconnected like channels.

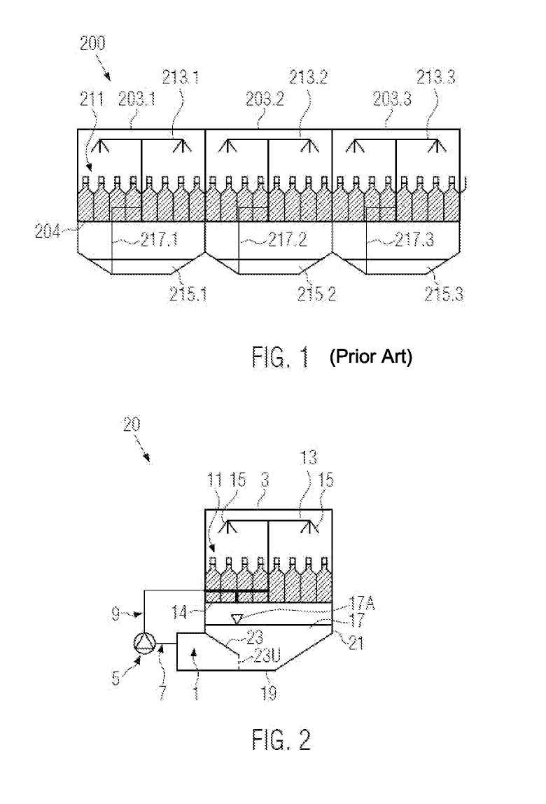

FIG. 2 shows a device 20 for the thermal treatment of products in containers 11 with a separation unit 1 according to the present disclosure. Only by way of example, a pasteurization device, pasteurizer, with only one treatment zone 3 is shown in FIG. 2 as a device 20. It will be understood, however, that the device may also comprise a heating device for containers 11, or a cooling device for containers 11. It will also be understood that the device 20 may comprise more than one treatment zone 3. One or several, in particular also all of the treatment zones 3 may comprise separation units 1.

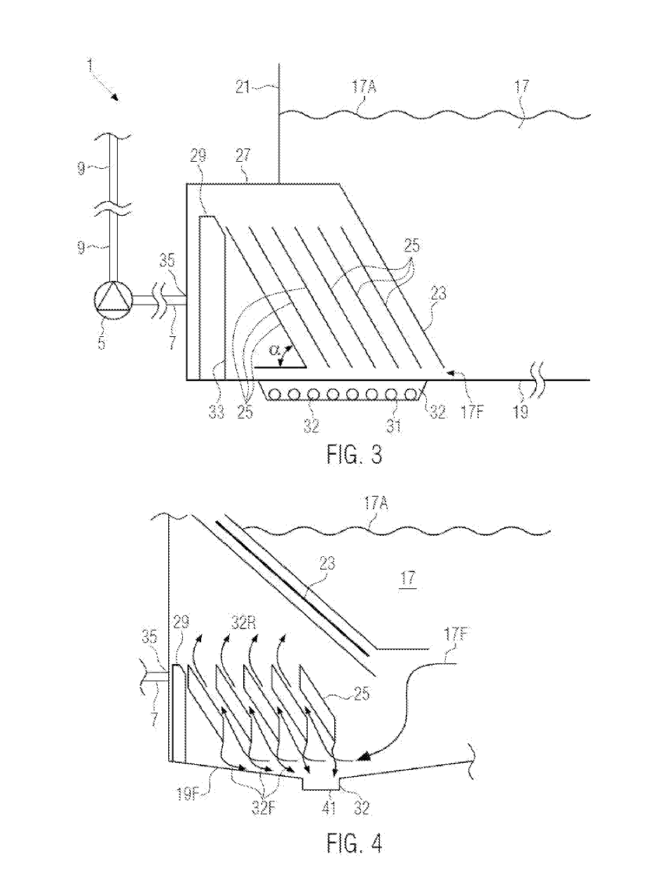

The treatment zone 3 shown in FIG. 2 shows an irrigation system 13 with nozzles 15 for spraying or irrigating the containers 11. Only by way of example, the containers 11 are sprayed or irrigated here from above. However, it is also possible to spray the containers, for example, from the side. As a process liquid 17 for irrigation or spraying, water may be used, for example. The water may typically contain chemical additives, for example to dissolve organic substances that swim in the water. The containers 11 are transported on a conveyor belt 14 in the treatment zone 3 of the device 20. The device 20 and/or the treatment zone 3 may include supply and discharge conveyors for containers (not shown here). In FIG. 2, a collecting tank 21 is shown underneath the conveyor belt 14. In the collecting tank 21, process liquid 17 with a liquid level 17A is shown. By way of example, a partition 23 is shown on the left side of the collecting tank 21 which ends above the bottom 19 of the collecting tank 21. So, the partition 23 does not reach completely to the bottom 19 of the collecting tank. Thereby, an opening 23U is formed through which the process liquid 17 may flow or stream into the device 1. The flowing or streaming of the process liquid 17 may be assisted by pumping or sucking up. From the separation unit 1, a connecting pipe 7 leads to a pump 5 by which a circulation of the process water/the process liquid 17 may be produced by pumping. It will be understood that only by way of example, only one pump 5 is shown in FIG. 2. However, several pumps may also be used. Equally, the pump may also be provided at another site in the circuit of the process water. It is also possible (not shown here) that a storage with fresh process liquid 17 is provided to balance any losses of process liquid 17. The circuit for the process liquid 17 is closed by a connecting pipe 9, and the process liquid 17 is again supplied to the irrigation system 13 for irrigation. The device may furthermore comprise heat exchangers etc., for example to bring the process liquid 17 to a certain temperature level by heating or cooling on its way between the separation unit 1 and the irrigation device 13. Heat exchangers possibly required for this are not shown. The separation unit 1 will be illustrated more in detail with reference to FIGS. 3 and 4.

FIG. 3 shows a side view of the separation unit 1 of FIG. 2 according to the present disclosure. In FIG. 3, the collecting tank 21 with the process liquid 17 and the liquid level 17A is shown in sections. The partition 23 of the separation unit 1 does not completely reach to the bottom 19 of the collecting tank 21. Arrow 17F indicates a direction of flow or stream of the process liquid 17. This flow or stream 17F of the process liquid 17 may be generated by the pump 5. The use of a suction device or a combined pump and suction device (not shown) is also possible. FIG. 3 shows several inclined lamellae 25 which are arranged essentially in parallel. The distance between the lamellae 25 is essentially constant. However, it is also possible to choose different distances or to choose different distances for the lamellae 25 by groups. In FIG. 3, only by way of example, six lamellae are represented. It will be understood, however, that a different number of lamellae may also be selected. The process liquid 17 flows along the lamellae 25. Via an overflow edge 29, the process liquid 17 flows to the pump 5. The process liquid 17 may leave the separation unit 1 only at the opening 35. From the opening 35, the process liquid 17 may flow through the pipe 7 to the pump 5 and from there via the pipe 9 again to a treatment zone of the device 20, as was discussed with reference to FIG. 1. The separation unit 1 has an upper limit which is provided with reference numeral 27. The overflow edge 29 is only by way of example represented above the end of the lamellae 25. However, it is also possible to select the upper level of the overflow edge 29 corresponding to the upper edges of the lamellae 25. The lamellae 25 typically have the same sizes/dimensions. In FIG. 3, the lamellae 25 are each mounted at the same height. This means that the lower and the upper ends of each lamella each have the same distances with respect to the bottom 19 of the collecting tank 21. Left of the lamellae 25, a separation edge 33 is provided which forms, together with the overflow edge 29, a separation of the lamellae 25 with respect to the outlet of the separation unit 1, i. e. the opening 35.

FIG. 3 furthermore shows a screen box 31. This is typically designed to be exchangeable. Moreover, particles 32 are shown which, due to gravity, may sediment downwards along the lamellae 25. Here, the term particle 32 may include, for example, broken glass, sand or minor metal particles. The screen box 31 may be emptied regularly, for example. While it is principally possible to omit a screen box such as the screen box 31, one would have to open the separation unit 1 after a certain operation period of the device 20 and clean the bottom of the separation unit 1. FIG. 3 furthermore shows that the lamellae 25 are provided at an angle a to the horizontal line. The angle a may be, for example, 30°<α<60° to support the sedimentation of the particles 32 under the influence of gravity along the surfaces of the lamellae 25.

FIG. 4 shows a schematic diagram with a perspective view of a separation unit 1 as it is also shown in FIG. 3. The same elements as in FIG. 3 are designated with the same reference numerals. Here, in particular the flow of the process liquid 17 is indicated by arrow 17F. This arrow indicates the flow or stream of process liquid 17 along the lamellae 25. With the arrows 32F, the sedimentation, i. e. the sinking of particles is indicated. At the upper edge of the lamellae, arrows 32R indicate a cleaned process liquid 17. Here, it will be understood that a cleaned process liquid 17 contains less particles than the liquid before it has flown over the sedimentation surfaces of the lamellae 25. In contrast to FIG. 3, the screen box 41 is in this figure narrower than the screen box 31 in FIG. 3. Thereby, the width of the screen box 41 in FIG. 4 covers fewer lamellae 25 than in FIG. 3. The bottom 19F, however, is drawn with a slight inclination, so that particles may slip into the screen box 41. As in FIG. 3, the screen box 41 in FIG. 4 is also exchangeable.

What is claimed is:

1. A device for thermally treating products in containers, comprising supply and discharge conveyors for containers; at least one treatment zone for one of irrigating and spraying the containers with a process liquid; a circulation circuit for at least partially reusing the process liquid, the circulation circuit comprising at least one pump; at least one separation unit with a plurality essentially parallel lamellae for cleaning the process liquid from particles; and, the pump pumping the process liquid along the lamella.

2. The device according to claim 1, wherein the lamellae are provided underneath the liquid surface and are completely wetted by the process liquid.

3. The device according to claim 1, wherein the lamellae are provided obliquely to the horizontal line at an angle a, where preferably 30°<α<60°.

4. The device according to claim 1, wherein the lamellae are one of exchangeable individually and in the form of lamella packs which comprise several lamellae.

5. The device according to claim 1, wherein the lamellae comprise an essentially liquid-tight surface.

6. The device according to claim 1, wherein the separation unit further comprises an exchangeable screen box at the bottom of the separation unit for collecting the particles.

7. The device according to claim 1, wherein the separation unit further comprises an overflow edge via which the cleaned process liquid gets into the circulation circuit again.

8. The device according to claim 1, and comprising a plurality of treatment zones for one of for irrigating and spraying the containers, wherein the at least one separation unit is associated with a plurality of the treatment zones.

9. The device according to claim 1, and comprising a plurality of treatment zones for one of irrigating and spraying containers, wherein one separation unit is associated with each treatment zone.

10. The device according to claim 1, wherein the device is one of a pasteurization device, a heating device, and a cooling device.

11. Method of using a separation unit for cleaning a process liquid from particles in a device for thermally treating products in containers, the device having supply and discharge conveyors for containers; at least one treatment zone for irrigating or spraying the containers with said process liquid; and a circulation circuit for at least partially reusing said process liquid, the circulation circuit comprising at least one pump; wherein
the separation unit comprises several, essentially parallel lamellae, the method including pumping said process liquid along the lamellae.

12. The device according to claim 1, wherein the process liquid comprises water.

13. The device according to claim 1, wherein the particles comprise one of broken glass, sand, and a combination thereof.

14. The device according to claim 3, wherein the angle a is greater than 30° and less than 60°.

15. The device according to claim 5, wherein the surface is coated.
